(19) United States
(12) Patent Application Publication
(10) Pub. No.: US 2020/0155636 A1
(43) Pub. Date: May 21, 2020

(54) Medicament and Beverage for Improving Joint and Bone Diseases

(71) Applicants: Carlo Bermes, Woerrstadt (DE); Joerg Eppelmann, Stadecken- Elsheim (DE)

(72) Inventors: Carlo Bermes, Woerrstadt (DE); Joerg Eppelmann, Stadecken- Elsheim (DE)

(21) Appl. No.: 16/630,184

(22) PCT Filed: Jul. 10, 2018
(86) PCT No.: PCT/EP2018/068656

§ 371 Date: Jan. 10, 2020

(30) Foreign Application Priority Data
Jul. 11, 2017 (DE) 10 2017 006 532.2

(51) Int. Cl. A61K 36/73; A61K 31/728; A61K 9/00; A61P 19/02

(52) U.S. Cl. CPC A61K 36/73; A61P 19/02; A61K 9/0095; A61K 31/728

(57) ABSTRACT

The invention relates to a medicament containing hyaluronic acid and natural polyphenol antioxidants for treating joint and bone diseases by oral administration, and to a beverage containing hyaluronic acid and natural polyphenol antioxidants for improving joint and bone diseases.

The invention relates to a medicament containing hyaluronic acid and natural polyphenol antioxidants for treating joint and bone diseases by oral administration, and to a beverage containing hyaluronic acid and natural polyphenol antioxidants for improving joint and bone diseases.

### BACKGROUND OF THE INVENTION

Hyaluronic acid is tipped as a wonder weapon in anti-aging. This endogenous substance is also employed in therapy for problems with joints. In the anti-aging treatment, hyaluronic acid is essentially supplied by topical application to the skin areas to be treated, in addition to oral applications by specific beverages containing collagen, hyaluronic acid, borage oil emulsion, and a glutamic acid derivative (EP-B-2532252), or hyaluronic acid, pomegranate juice extract, cocoa flower syrup, vitamin C, and concentrates of carrot and safflower (Proceanis®). To date, the treatment of joint and bone diseases has been effected only by injection into the joint to be treated, or at the bone to be treated, which is not unlikely to result in complications, such as inflammations. Furthermore, medicaments, beverages and food supplements are known in the art which contain hyaluronic acid in combination with: small amounts of polyphenols, suitable for the treatment and prevention of certain diseases, including osteoarthritis and osteoporosis (JP 2015 038045); grape juice, green or black tea, suitable for the treatment of osteoarthritis (US 2004/0198695); antioxidants such as green tee extract, polyphenols and/or bioflavonoids suitable for the treatment of osteoporosis (US 2006/0239987); bioflavonoids and antioxidants, suitable for the treatment of osteoporosis (WO 2008/152015).

It has now been found that a specific beverage containing hyaluronic acid in combination with a high amount of natural polyphenol antioxidants, which was actually developed for anti-aging treatment, resulted in significant improvements when ingested by persons having joint or bone diseases. Thus, when a 250 ml unit of the beverage with 200 mg of hyaluronic acid per day was ingested by the test subjects, a significant improvement of the symptoms could be seen already after 2-4 days, i.e., only a little or no pain at all, and improvement of mobility.

### SUMMARY OF THE INVENTION

Thus, the invention relates to

(1) a medicament containing hyaluronic acid for oral application in the treatment of joint and bone diseases;
(2) a preferred embodiment of the medicament (1), in which said medicament further contains natural polyphenol antioxidants, and said medicament preferably has a content of natural polyphenol antioxidants that corresponds to an oxygen radical absorption capacity (ORAC) of at least 700 μmol trolox equivalents (TE) per 100 ml of the medicament;
(3) a beverage for improving joint and bone diseases, containing hyaluronic acid and natural polyphenol antioxidants, the content of natural polyphenol antioxidants in said beverage corresponding to an ORAC of at least 700 μmol TE per 100 ml of the beverage;
(4) a composition containing hyaluronic acid and natural polyphenol antioxidants, the content of natural polyphenol antioxidants in said composition corresponding to an ORAC of at least 700 μmol TE per 100 ml of the composition, for use in the treatment of joint and bone diseases by oral application; and
(5) a method for the treatment of joint and bone diseases, which comprises orally administering a subject in need of such a treatment a composition containing hyaluronic acid and natural polyphenol antioxidants, the content of natural polyphenol antioxidants in said composition corresponding to an ORAC of at least 700 μmol TE per 100 ml of the composition.

### DETAILED DESCRIPTION OF THE INVENTION

Here is a brief survey of hyaluronic acid itself. Hyaluronic acid is produced by the body itself. Up to an age of 30 years, the production is usually at 100%, at 40, it is only 50%, and from an age of 60 years, it is only about 10% of the original production by the body. This is just where the medicament, composition and beverage according to the invention come in, i.e., to provide the body with the lacking hyaluronic acid in order to support the body thereby. The body then distributes the hyaluronic acid to the sites where it is necessary, as provided by nature. The scientific community currently discusses the effectiveness of hyaluronic acid. It has definitely been proven that hyaluronic acid survives the gastric acid and is absorbed in the small intestine. The exact mechanisms of action are still being examined. Hyaluronic acid is suspected to be transported, after absorption in the body, to the corresponding sites in the body where there is a hyaluronic acid deficiency.

In scientific studies, the mechanism of action of hyaluronic acid in oral application has already been described, and it was found that only molecular weights of at most 500 kDa can be absorbed by the small intestine at all. In the medicament or beverage according to the invention, a hyaluronic acid having a molecular weight of from 50 to 200 kDa is preferably used, so that a very good absorption is ensured. The content of hyaluronic acid in the medicament, composition or beverage according to the invention is preferably from 10 to 2000 mg/l, preferably from 100 to 1500 mg/l, which corresponds to a hyaluronic acid daily dose of 1-500 mg, preferably 20-300 mg (for a unit dose of 200 mg).

Another possible cause of the effectiveness of hyaluronic acid for treating joint and bone diseases and the necessary transport to the corresponding sites in the body, without being bound to theory, may also be due to the common administration of hyaluronic acid with a sufficient amount of polyphenol antioxidants, which are contained in the medicament, composition and beverage according to the invention. Suitable sources of such natural polyphenols include, for example, chokeberry (genus Aronia; including chokeberry, black chokeberry and purple chokeberry, A. arbutifolia, A. melanocarpa and A. prunifolia, respectively/with an ORAC value of 16,062 μmol of trolox equivalent (TE)/100 g), cranberry (9,090 μmol TE/100 g), plum (6,100 μmol TE/100 g), raspberry (5,065 μmol TE/100 g), blueberry (4,696 μmol TE/100 g), and pomegranate (4,479 μmol TE/100 g) (source: US Department of Agriculture, Database for the Oxygen Radical Absorbance Capacity (ORAC) of selected foods, 2010), and the juices and extracts derived therefrom. Particularly preferred within the meaning of the present invention is the use of chokeberry juice, which has an ORAC value of approximately 7,000 μmol TE/100 g. Preferably, the medicament has an ORAC value of at least 700 μmol TE/100 ml, preferably at least 1200 μmol TE/100 ml, more preferably at least 1500 μmol TE/100 ml (the hyaluronic acid beverage Proceanis® with 5% pomegranate juice extract, which is already commercially available, has an ORAC value of at most 150 μmol TE/100 ml). More preferably, the medicament or beverage preferably contains at least 10% by weight, preferably at least 15% by weight, chokeberry juice.

In addition, the medicament/beverage may further contain pharmaceutically or dietetically acceptable carrier fluids, thickening agents, stabilizers, salts, vitamins, sweeteners, and/or flavoring agents. There may be mentioned, in particular, water and other fruit juices, such as orange or grape juice. In particular, the beverage according to the invention contains 40% by volume water, 40% by volume grape juice, 20% by volume chokeberry juice, and 200 mg of hyaluronic acid with a molecular weight of 50-200 kDa.

The joint and bone diseases treated within the meaning of the present invention include joint pain, arthrosis, joint inflammations, osteoarthritis and osteoporosis, and all kinds of sports injuries. The administration of the medicament/beverage according to the invention leads to a significant improvement, i.e., increased mobility and load capacity of the joint, is observed already after a short treatment period.

The invention is further illustrated by means of the following Example.

### Example

24 test subjects suffering from joint diseases (knee, ankle, shoulder, fingers (gout), back, wrist, elbow) drank 250 ml per day over 24 days of a beverage containing hyaluronic acid, consisting of 40% by volume water, 40% by volume grape juice, 20% by volume chokeberry juice, and 200 mg of hyaluronic acid with a molecular weight of 50-200 kDa. A significant improvement of the pain condition and mobility was observed in all test subjects already after 2-4 days.

1.-18. (canceled)

19. A composition or medicament containing
(a) hyaluronic acid and
(b) one or more chokeberry juice-derived polyphenol antioxidants with a polyphenol antioxidant content in said medicament that corresponds to an oxygen radical absorption capacity (ORAC) of at least 1200 μmol trolox equivalents (TE) per 100 ml of the medicament,
for the treatment of joint and bone diseases by oral application.

20. The composition or medicament of claim 19, wherein said hyaluronic acid (a) has a molecular weight of up to 500 kDa.

21. The composition or medicament of claim 20, wherein said hyaluronic acid (a) has a molecular weight from 50 to 200 kDa.

22. The composition or medicament of claim 19, wherein the content of hyaluronic acid in the composition or medicament is from 10 to 2000 mg/1, which corresponds to a hyaluronic acid daily dose of 1 to 500 mg.

23. The composition or medicament of claim 22, wherein the content of hyaluronic acid in the composition or medicament is from 100 to 1500 mg/1, which corresponds to a hyaluronic acid daily dose of 20 to 300 mg.

24. The composition or medicament of claim 19, wherein said composition or medicament contains at least 10% by volume, or at least 15% by volume, of chokeberry juice.

25. The composition or medicament of claim 19, further containing pharmaceutically acceptable carrier fluids, thickening agents, stabilizers, and/or flavoring agents.

26. The composition or medicament of claim 19, containing 40% by volume water, 40% by volume grape juice, 20% by volume chokeberry juice, and 200 mg of hyaluronic acid.

27. A beverage for improving joint and bone diseases, containing
(a) hyaluronic acid and
(b) one or more chokeberry juice-derived polyphenol antioxidants with a polyphenol antioxidant content in said beverage corresponding to an ORAC of at least 1200 μmol TE per 100 ml of the beverage.

28. The beverage of claim 27, wherein said hyaluronic acid has a molecular weight of up to 500 kDa.

29. The beverage of claim 28, wherein said hyaluronic acid has a molecular weight from 50 to 200 kDa.

30. The beverage of claim 27, wherein the content of hyaluronic acid in the beverage is from 10 to 2000 mg/l.

31. The beverage of claim 30, wherein the content of hyaluronic acid in the beverage is from 100 to 1500 mg/l.

32. The beverage of claim 27, wherein the beverage contains at least 10% by volume, or at least 15% by volume, of chokeberry juice.

33. The beverage of claim 27, further containing dietetically acceptable carrier fluids, thickening agents, stabilizers, and/or flavoring agents.

34. The beverage of claim 27, containing 40% by volume water, 40% by volume grape juice, 20% by volume chokeberry juice, and 200 mg of hyaluronic acid.

35. A method for the treatment of joint and bone diseases, which comprises orally administering a subject in need of such a treatment a composition containing
(a) hyaluronic acid and
(b) one or more chokeberry juice-derived polyphenol antioxidants with a natural polyphenol antioxidant content in said composition that corresponds to an ORAC of at least 1200 μmol TE per 100 ml of the composition.

36. The method of claim 35, wherein said hyaluronic acid has a molecular weight of up to 500 kDa.

37. The method of claim 36, wherein said hyaluronic acid has a molecular weight from 50 to 200 kDa.

38. The method of claim 35, wherein the content of hyaluronic acid in the composition is from 10 to 2000 mg/l.

39. The method of claim 38, wherein the content of hyaluronic acid in the composition is from 100 to 1500 mg/l.

40. The method of claim 35, wherein the composition contains at least 10% by volume, or at least 15% by volume, of chokeberry juice.

41. The method of claim 35, wherein the composition further contains pharmaceutically acceptable carrier fluids, thickening agents, stabilizers, and/or flavoring agents.

42. The method of claim 35, wherein the composition contains 40% by volume water, 40% by volume grape juice, 20% by volume chokeberry juice, and 200 mg of hyaluronic acid.
